(19) United States
(12) Patent Application Publication
(10) Pub. No.: US 2023/0404641 A1
(43) Pub. Date: Dec. 21, 2023

(54) SURGICAL SMOKE EVACUATION DEVICE

(71) Applicant: David Louis Kirschman, Dayton, OH (US)

(72) Inventor: David Louis Kirschman, Dayton, OH (US)

(73) Assignee: Aerobiotix, LLC, Miamisburg, OH (US)

(21) Appl. No.: 18/318,848

(22) Filed: May 17, 2023

Related U.S. Application Data
(60) Provisional application No. 63/343,346, filed on May 18, 2022.

(51) Int. Cl. A61B 18/00; A61B 46/10

(52) U.S. Cl. CPC A61B 18/00; A61B 2218/008; A61B 46/10

(57) ABSTRACT

An air-surface disinfector has a conformable tube member that is detachably mounted to a vacuum generator. The conformable tube member has an integral filter housing or support for receiving and housing at least one or plurality of filters or other decontamination apparatus or means. The vacuum generator comprises a cavity that is defined by a plurality of walls formed in a predetermined configuration that complements the shape of the filter housing or support so that the filter housing or support can be dropped into the cavity, thereby securing the conformable tube member to the vacuum generator. The conformable tube member may optionally comprise a sterile sheath that is integral therewith or detachably mounted thereon. The conformable tube member and associated filter housing or support are disposable and may be disposed of after a predetermined time or after a predetermined contamination has been achieved.

### CROSS-REFERENCE TO RELATED APPLICATIONS

The present application claims priority to provisional U.S. Application Ser. No. 63/343,346, filed May 18, 2022, to which Applicant claims the benefit of the earliest filing date of this application, which is incorporated herein by reference and made a part hereof.

### BACKGROUND OF THE INVENTION

### 1. Field of the Invention

Surgical procedures often incorporate the use of various energy-driven cautery and tissue dissection instruments which thermally ablate human tissue. A significant drawback of these systems is the generation of a surgical smoke plume. This smoke contains hazardous compounds including particulates, volatile organic compounds, toxic gases, and pathogenic microorganisms. This smoke plume contaminates the operating room air, which is detrimental to the occupational health of the surgical staff and patient.

### 2. Description of the Related Art

The prior art describes multiple systems for the evacuation of a surgical smoke plume. However, these systems have significant human factor drawbacks. In prior art evacuation systems, a sterilized suction instrument needs to be placed directly into the surgical field. These devices include cautery pencils with integral vacuum lumens, suction handpieces, or surgical tubing. In each case, the suction device alters the visual and/or tactile access to the surgical site. This alteration can be detrimental to the surgeon, resulting in reduced visualization, access, and tactile feedback from the surgical site.

What is needed, therefore, is a surgical smoke evacuation device which is proximate to but does not intrude upon the surgical site.

### SUMMARY OF THE INVENTION

In the current invention, a sterile self-supporting conformable tube is placed in proximity to the surgical site but positioned outside of the surgeon's line of sight. In order to safely and effectively accomplish this, several innovations are required. These include sterilization means for the self-supporting tube, means to isolate the tube and associated filter from a vacuum system, integration of a filtration means, and a means to incorporate different tube shapes for different anatomical needs/positions. These and further innovations shall be elucidated in the claims and drawings below.

One object of the invention is to provide a sterile, self-supporting conformable tube that is detachable and removable from a vacuum unit.

Another object of one embodiment is to provide a conformable tube member detachably mounted to a vacuum unit.

Still another object is to provide a means for isolating a conformable tube and an associated filter from the vacuum system or vacuum generator.

Yet another object of the invention is to provide a self-supporting conformable tube and be detachably mounted to a vacuum unit and that itself contains at least one or a plurality of filters or decontamination apparatus or means.

Still another object of one embodiment is to provide a detachable replaceable conformable tube that houses at least one filter or decontamination apparatus or means.

Another object of the embodiment being described is to provide a conformable tube that can be adapted and changed to accommodate a particular surgical procedure. Such dimensions may comprise an overall length, an internal lumen or passageway dimension or the like.

Yet another object is to provide a conformable tube that may have an integral or replaceable nozzle, the nozzle shape selected from a family of nozzles and whose nozzle shape can be selected depending upon the surgical procedure to be performed.

Yet another object of the embodiments being described is a system that comprises and incorporates a plurality of different tube shapes for different anatomical needs or positions.

Another object is to provide a conformable tube member and integrated filter assembly that can be replaced upon demand by simply replacing the conformable tube member.

In one aspect, one embodiment of the invention comprises a surgical smoke evacuation system for evacuating smoke or vapor from a surgical site associated with a patient, the surgical smoke evacuation system comprising a vacuum generator for generating a vacuum, a conformable tube member having a first end having an opening or nozzle for entry or passage of surgical smoke or vapor generated during a surgical procedure, the conformable tube member also having a second end having a filter housing or support adapted to receive and to treat the surgical smoke or vapor passing between the first end and the second end and through the conformable tube member, and at least one of the first end or the second end being removably mounted to the vacuum generator such that the opposite end may be positioned in proximity to the surgical site.

In another aspect, another embodiment of the invention comprises a disposable surgical smoke evacuation tube for use with a surgical smoke evacuation system having a vacuum generator, the disposable surgical smoke evacuation tube comprising a conformable tubular body member having a first end and a second end, at least one filter housing or support for housing at least one filter, the at least one filter housing or support being coupled to and in fluid communication with the conformable tubular body member, the conformable tubular body member being adapted and shaped to be removably secured or mounted to a vacuum generator receiving area of the vacuum generator such that it is stable so that a user can position the filter housing or support at the receiving area so that the vacuum generator can generate a vacuum through the conformable tubular body member so that at least one of the first or second ends can be positioned in proximity to a surgical site to evacuate smoke and vapor from the surgical site associated with a patient.

In still another aspect, another embodiment of the invention comprises a surgical smoke evacuation system comprising a conformable self-supporting tube with a first tube end comprising an opening or nozzle for the entry of surgical smoke, the first tube end further comprising filtration means or filter, the filtration means or filter being in an operative relationship with the opening or nozzle, the self-supporting tube comprising a second tube end dimensioned for attachment to at least one of a vacuum and or filtration means.

In still another aspect, another embodiment of the invention comprises a surgical smoke evacuation system comprising a self-supporting and conformable tube with a first tube end comprising an opening or nozzle for the entry of surgical smoke, a second tube end contiguous and substantially integral to a filter housing or support for treatment of surgical smoke, the filter housing or support having at least one external airflow aperture, a vacuum unit comprising a vacuum generation or transmission means, separate from the self-supporting and conformable tube and the filter housing or support, the vacuum unit comprising connection and control means to connect to the filter housing or support and the aperture.

This invention, including all embodiments shown and described herein, could be used alone or together and/or in combination with one or more of the features covered by one or more of the following list of features:

The surgical smoke evacuation system wherein the vacuum generator comprises a vacuum generator housing having a filter housing or support receiving area adapted and sized to mate with and receive the filter housing or support.

The surgical smoke evacuation system wherein the filter housing or support receiving area comprises a vacuum generator predetermined shape for removably receiving and supporting the filter housing or support such that the conformable tube member can be conformed or moved to enable the first end to be moved or conformed to a proximity at or near the surgical site.

The surgical smoke evacuation system wherein the filter housing or support has a filter housing or support predetermined shape that generally complements the vacuum generator predetermined shape so that the filter housing or support can be received and stored therein so that the conformable tube member can be detachably mounted to the vacuum generator.

The surgical smoke evacuation system wherein the vacuum generator predetermined shape defines a filter housing or support receiving area for receiving the filter housing or support, the filter housing or support having a predetermined filter housing or support shape that generally complements a shape of the filter housing or support so that after the filter housing or support is received in the filter housing or support receiving area, the conformable tube member is in fluid communication with the vacuum generator so that the vacuum generator can cause a negative pressure through the conformable tube member so that the smoke and vapor may be collected through the first end after the first end has been placed in proximity to the surgical site by a user.

The surgical smoke evacuation system wherein the at least one of the vacuum generator or the conformable tube member comprise at least one securing means or lock for securing or locking, respectively, filter housing or support onto the vacuum generator.

The surgical smoke evacuation system wherein the vacuum generator predetermined shape is sized and dimensioned to receive the filter housing or support such that it is secure and stabilized during movement of the conformable tube member.

The surgical smoke evacuation system wherein the vacuum generator comprises a vacuum generator housing having a filter housing or support receiving area for receiving the filter housing or support.

The surgical smoke evacuation system wherein the vacuum generator predetermined shape is at least one of a cylinder, polyhedron or is pyramidal.

The surgical smoke evacuation system wherein the conformable tube member is reshapable and conformable by the user so that at least one of the first end or the second end can be situated in proximity to the surgical site, the conformable tube member having memory and being self-supporting so that it remains in position after the user has positioned the first end.

The surgical smoke evacuation system wherein the filter housing or support has a filter housing or support predetermined shape that generally complements a vacuum generator predetermined shape so that the filter housing or support can be received therein so that the conformable tube member can be detachably mounted to the vacuum generator.

The surgical smoke evacuation system wherein the vacuum generator has a vacuum generator controller, the filter housing or support comprises communication means or a filter housing or support control for communicating data to and/or from the vacuum generator controller regarding a state or condition data of the at least one filter, the state or condition data comprising data relating to at least one of usage time, filter expiration or usage factors.

The surgical smoke evacuation system wherein the filter housing or support comprises an air sensor coupled to the vacuum generator controller for sensing a plurality of parameters, the plurality of parameters comprising carbon dioxide, volatile organic compound, particulate, pressure, temperature, humidity and/or other physical parameters.

The surgical smoke evacuation system wherein the conformable tube member further comprises a sterile flexible drape or sheath situated on the conformable tube member.

The surgical smoke evacuation system wherein the conformable tube member further comprises a sterile flexible drape or sheath situated on the conformable tube member, the sterile flexible drape or sheath being removably mounted on the conformable tube member so that it can be removed and discarded and replaced with another one of the sterile flexible drape or sheath.

The surgical smoke evacuation system wherein the filter housing or support may comprise a predetermined shape that is at least one of a t cylinder, polyhedron or is pyramidal.

The surgical smoke evacuation system wherein the first end is an inlet end, the filter housing or support being situated on the inlet end.

The surgical smoke evacuation system wherein the conformable tube member can be reversibly re-shaped by the user and will substantially remain in the shape, allowing for placement proximate to a surgical site, without requiring direct patient contact, a self-supporting tube mechanism or mechanisms integral to and/or acting upon the tube described by any of hinged, pinned, segmented, friction, jointed, telescoping, malleable, sprung, elastic, conformable, having single segment or plurality of homogeneous or heterogeneous segments, of various dimensions, materials, theories of operation, axes of motion, ranges of motion, geometries, rotations, angles, mechanisms, locks, clutches, power sources, automations, motors, gears, drives, servos, sensors, pistons, actuators, splines, chains, belts, cables, tracks, control mechanisms, and any other ways or means resulting in self-supporting conformational change of the tube by the user, while maintaining a substantially contiguous internal lumen.

The surgical smoke evacuation system wherein the conformable tube member can be reversibly re-shaped by the user and will substantially remain in the shape, allowing for placement proximate to a surgical site, without requiring direct patient contact, a self-supporting tube mechanism or mechanisms integral to and/or acting upon the tube resulting in self-supporting conformational change of the tube by the user, while maintaining a substantially contiguous internal lumen.

The surgical smoke evacuation system wherein the inlet end comprises a nozzle.

The surgical smoke evacuation system wherein the self-supporting tube mechanism and the filter housing or support comprise a substantially integrated assembly which can be handled or replaced a single unit, the tube and the housing further comprising a contiguous air flow channel, the channel comprising a lumen being substantially air and watertight along its length from the first tube opening to the filter housing or support and through an airflow aperture.

The surgical smoke evacuation system wherein the filter housing or support comprises an air filtration means, including HEPA, ULPA, granular, chemical, catalytic, electrostatic, or similar means to affect air decontamination.

The surgical smoke evacuation system wherein the vacuum incorporates an air sensing means, the air sensing means comprising modalities including carbon dioxide, volatile organic compound, particulate, pressure, temperature, humidity and/or other physical parameters, the parameters feeding back into the air sensing means for display, memory, or transmission.

The surgical smoke evacuation system wherein the conformable tube member comprises a sterile flexible external drape or sheath.

The surgical smoke evacuation system wherein the conformable tube member is manufactured from a re-sterilizable metallic or polymeric material, such as stainless steels, polyethylenes, polysulphones, silicones, and/or acetals for autoclave sterilization.

The surgical smoke evacuation system wherein the conformable tube member comprises a detachable distal nozzle, which can be sized or shaped to anatomical requirements of a specific surgical procedure.

The disposable surgical smoke evacuation tube wherein the conformable tubular body member has memory so that at least one of the first or second end can be positioned placed in proximity to the surgical site and remain conformed in a desired location.

The disposable surgical smoke evacuation tube wherein the at least one filter housing or support is located on at least one of the first or second ends and in fluid communication with the tubular body member.

The disposable surgical smoke evacuation tube wherein the at least one filter housing or support is situated on an outlet end of the conformable tubular body member, the at least one filter housing or support having a predetermined filter housing or support shape, the vacuum generator having a vacuum generator receiving area that is sized, shaped and adapted to generally complement the shape of the predetermined filter housing or support shape so that the at least one filter housing or support can be situated in the vacuum generator receiving area and coupled to the vacuum generator so that an inlet end of the conformable tubular body member may be positioned at or near the surgical site.

The disposable surgical smoke evacuation tube wherein the vacuum generator receiving area is defined by a plurality of walls sized and dimensioned and configured to mate with and receive the filter housing or support such that it is secure and stabilized during movement of the first end.

The disposable surgical smoke evacuation tube wherein the predetermined filter housing or support shape is at least partially cylindrical, polyhedron or pyramidal in shape.

The disposable surgical smoke evacuation tube wherein the predetermined filter housing or support shape and the vacuum generator receiving area are both at least partially cylindrical, polyhedron or pyramidal and complementary in the shape so that the at least one filter housing or support can be received and secured in the receiving area, thereby coupling the tubular body member to the vacuum generator.

The disposable surgical smoke evacuation tube wherein the conformable tubular body member comprises a sterile protective cover or sheath.

The disposable surgical smoke evacuation tube wherein the sterile protective cover or sheath is both removeable and disposable.

The disposable surgical smoke evacuation tube wherein at least one of the vacuum generator or the tubular body member comprises at least one securing means or lock for securing or locking, respectively, at least one filter housing or support to the vacuum generator.

The disposable surgical smoke evacuation tube wherein the conformable tubular body member is re-shapable and conformable by the user so that an inlet end can be situated in proximity to a surgical site, the conformable tubular body member having memory and being self-supporting so that it remains in position after the user has positioned or conformed the first end in proximity to the surgical site.

The disposable surgical smoke evacuation tube wherein the at least one filter housing or support is situated on and in fluid communication with the first end.

The disposable surgical smoke evacuation tube wherein the vacuum generator comprises a vacuum generator controller, the filter housing or support comprising a data collector or collection means for collecting usage data regarding the filter and for communicating the usage data to and/or from the vacuum generator controller regarding a state or condition data of the at least one filter, the state or condition data comprising data relating to at least one of usage time, filter expiration or usage factors.

The disposable surgical smoke evacuation tube wherein the conformable tubular body member can be reversibly re-shaped by the user and will substantially remain in the shape, allowing for placement proximate to a surgical site, without requiring direct patient contact, a self-supporting tube mechanism or mechanisms integral to and/or acting upon the tube described by any of hinged, pinned, segmented, friction, jointed, telescoping, malleable, sprung, elastic, conformable, having single segment or plurality of homogeneous or heterogeneous segments, of various dimensions, materials, theories of operation, axes of motion, ranges of motion, geometries, rotations, angles, mechanisms, locks, clutches, power sources, automations, motors, gears, drives, servos, sensors, pistons, actuators, splines, chains, belts, cables, tracks, control mechanisms, and any other ways or means resulting in self-supporting conformational change of the tube by the user, while maintaining a substantially contiguous internal lumen.

The disposable surgical smoke evacuation tube wherein the conformable tube member can be reversibly re-shaped by the user and will substantially remain in the shape, allowing for placement proximate to a surgical site, without requiring direct patient contact, a self-supporting tube mechanism or mechanisms integral to and/or acting upon the tube resulting in self-supporting conformational change of the tube by the user, while maintaining a substantially contiguous internal lumen.

The disposable surgical smoke evacuation tube wherein the surgical smoke evacuation system comprises a plurality of conformable tubes having different shapes, at least one of which is selected for different anatomical needs or situations.

The disposable surgical smoke evacuation tube wherein the conformable tubular body member can be reversibly re-shaped by the user and will substantially remain in the shape, allowing for placement proximate to a surgical site, without requiring direct patient contact, a self-supporting tube mechanism or mechanisms integral to and/or acting upon the tube described by any of hinged, pinned, segmented, friction, jointed, telescoping, malleable, sprung, elastic, conformable, having single segment or plurality of homogeneous or heterogeneous segments, of various dimensions, materials, theories of operation, axes of motion, ranges of motion, geometries, rotations, angles, mechanisms, locks, clutches, power sources, automations, motors, gears, drives, servos, sensors, pistons, actuators, splines, chains, belts, cables, tracks, control mechanisms, and any other ways or means resulting in self-supporting conformational change of the tube by the user, while maintaining a substantially contiguous internal lumen.

The disposable surgical smoke evacuation tube wherein the conformable tube member can be reversibly re-shaped by the user and will substantially remain in the shape, allowing for placement proximate to a surgical site, without requiring direct patient contact, a self-supporting tube mechanism or mechanisms integral to and/or acting upon the tube resulting in self-supporting conformational change of the tube by the user, while maintaining a substantially contiguous internal lumen.

The disposable surgical smoke evacuation tube wherein an inlet end comprises a nozzle.

The surgical smoke evacuation system wherein the filtration means or filter comprises filtration media having at least one of a filter, a holder, an enclosure housing, textile, granules, or energy-based smoke treatment means or ultraviolet light (UV) treatment.

The surgical smoke evacuation system wherein the opening or nozzle comprises an inlet nozzle dimensioned to be removable and/or replaceable from the first tube end and further comprising a mechanical securing means comprising at least one of a clip, fastener, magnet, compression, friction, nesting or similar means.

The surgical smoke evacuation system wherein the first end is an inlet end, the filter housing or support being situated on the inlet end.

The surgical smoke evacuation system wherein the conformable tube can be reversibly re-shaped by the user and will substantially remain in the shape, allowing for placement proximate to a surgical site, without requiring direct patient contact, a self-supporting tube mechanism or mechanisms integral to and/or acting upon the tube described by any of hinged, pinned, segmented, friction, jointed, telescoping, malleable, sprung, elastic, conformable, having single segment or plurality of homogeneous or heterogeneous segments, of various dimensions, materials, theories of operation, axes of motion, ranges of motion, geometries, rotations, angles, mechanisms, locks, clutches, power sources, automations, motors, gears, drives, servos, sensors, pistons, actuators, splines, chains, belts, cables, tracks, control mechanisms, and any other ways or means resulting in self-supporting conformational change of the tube by the user, while maintaining a substantially contiguous internal lumen.

The surgical smoke evacuation system wherein the conformable tube can be reversibly re-shaped by the user and will substantially remain in the shape, allowing for placement proximate to a surgical site, without requiring direct patient contact, a self-supporting tube mechanism or mechanisms integral to and/or acting upon the tube resulting in self-supporting conformational change of the tube by the user, while maintaining a substantially contiguous internal lumen.

The surgical smoke evacuation system wherein the inlet end comprises a nozzle.

The disposable surgical smoke evacuation tube wherein a predetermined filter housing or support shape and a vacuum generator receiving area are both at least partially at least partially cylindrical, polyhedron or pyramidal and complementary in the shape so that the at least one filter housing or support can be received and secured in the vacuum generator receiving area, thereby coupling the conformable self-supporting tube to the vacuum generator.

The disposable surgical smoke evacuation tube wherein the conformable self-supporting tube comprises a sterile protective cover or sheath.

The disposable surgical smoke evacuation tube wherein the sterile protective cover or sheath is both removeable and disposable.

The disposable surgical smoke evacuation tube wherein at least one of the vacuum generator or the conformable self-supporting tube comprises at least one securing means or lock for securing or locking, respectively, at least one filter housing or support to the vacuum generator.

The disposable surgical smoke evacuation tube wherein the conformable self-supporting tube is re-shapable and conformable by the user so that the inlet end can be situated in proximity to a surgical site, the conformable tube having memory and being self-supporting so that it remains in position after the user has positioned or conformed the first end in proximity to the surgical site.

The disposable surgical smoke evacuation tube wherein the at least one filter housing or support is situated on and in fluid communication with the first end.

The disposable surgical smoke evacuation tube wherein the vacuum generator comprises a vacuum generator controller, the filter housing or support comprising a data collector or collection means for collecting usage data regarding the filter and for communicating the usage data to and/or from the vacuum generator controller regarding a state or condition data of the at least one filter, the state or condition data comprising data relating to at least one of usage time, filter expiration or usage factors.

The surgical smoke evacuation system wherein the self-supporting and conformable tube can be reversibly re-shaped by the user and will substantially remain in the shape, allowing for placement proximate to a surgical site, without requiring direct patient contact, a self-supporting tube mechanism or mechanisms integral to and/or acting upon the self-supporting and conformable tube described by any of hinged, pinned, segmented, friction, jointed, telescoping, malleable, sprung, elastic, conformable, having single segment or plurality of homogeneous or heterogeneous segments, of various dimensions, materials, theories of operation, axes of motion, ranges of motion, geometries, rotations, angles, mechanisms, locks, clutches, power sources, automations, motors, gears, drives, servos, sensors, pistons, actuators, splines, chains, belts, cables, tracks, control mechanisms, and any other ways or means resulting in self-supporting conformational change of the self-supporting and conformable tube by the user, while maintaining a substantially contiguous internal lumen.

The surgical smoke evacuation system wherein the self-supporting and conformable tube and the filter housing or support comprise a substantially integrated assembly which can be handled or replaced a single unit, the self-supporting and conformable tube and the filter housing or support further comprising a contiguous air flow channel, the channel comprising a lumen being substantially air and water tight along its length from the first tube opening to the filter housing or support and through the airflow aperture.

The surgical smoke evacuation system wherein the filter housing or support comprises an air filtration means, including HEPA, ULPA, granular, chemical, catalytic, electrostatic, or similar means to affect air decontamination.

The surgical smoke evacuation system wherein the filter housing or support comprises an electromagnetic or radiofrequency means to communicate data unidirectionally or bidirectionally between the self-supporting and conformable tube and/or the filter housing or support and the control means. the data comprising timing, filter expiration, or other usage factors.

The surgical smoke evacuation system wherein the vacuum unit incorporates an air sensing means, the air sensing means comprising modalities including carbon dioxide, volatile organic compound, particulate, pressure, temperature, humidity and/or other physical parameters, the parameters feeding back into the control means for display, memory, or transmission.

The surgical smoke evacuation system wherein the self-supporting and conformable tube comprises a sterile flexible external drape or sheath.

The surgical smoke evacuation system wherein the self-supporting and conformable tube is manufactured from a re-sterilizable metallic or polymeric material, such as stainless steels, polyethylenes, polysulphones, silicones, and/or acetals for autoclave sterilization.

The surgical smoke evacuation system wherein the vacuum unit comprises a substantial concavity dimensioned to receive and/or stabilize the filter housing or support.

The surgical smoke evacuation system wherein the vacuum unit comprises a securing means to lock the filter housing or support into position within a concavity, the securing means comprising a mechanical or magnetic interface.

The surgical smoke evacuation system wherein the self-supporting and conformable tube comprises a detachable distal nozzle, which can be sized or shaped to the anatomical requirements of a specific surgical procedure.

These and other objects and advantages of the invention will be apparent from the following description, the accompanying drawings, and the appended claims.

### BRIEF DESCRIPTION OF THE ACCOMPANYING DRAWINGS

FIG. 1 is a perspective view illustrating a drop in conformable tube member dropped into and detachably mounted from a vacuum generator;

FIG. 2 is a view of the embodiment shown in FIG. 1 with a fragmentary view, details of a filter housing or support assembly having a filter therein and showing a cavity in the vacuum generator or receiving the filter housing or support and thereby supporting the conformable tube member on the vacuum generator;

FIG. 3 is a view similar to FIG. 2 illustrating further details including a lock or latch that may be utilized to secure the conformable tube member to the vacuum generator;

FIGS. 4A-4B illustrate details of the cavity in the vacuum generator (FIG. 4A) and the mating filter housing or support (FIG. 4B);

FIGS. 5A-5B illustrate another embodiment showing a conformable tube member having a plurality of segments to permit the conformable tube member to be conformed to any predetermined position or shape.

FIGS. 6A-6B are another embodiment illustrating a conformable tube member coupled to an intermediate tube section;

FIGS. 7A-7B illustrate still another embodiment illustrating a conformable tube member coupled to a plurality of intermediate tube sections;

FIGS. 8A-8B illustrate the conformable tube member having a sterile drape or sheath and illustrating the nozzle being conformed from one position to another;

FIG. 9 is a view of another embodiment illustrating the filter housing or support situated on an inlet and the conformable tube member; and

FIGS. 10A-10B illustrate different embodiments of different nozzle shapes.

### DETAILED DESCRIPTION OF THE PREFERRED EMBODIMENTS

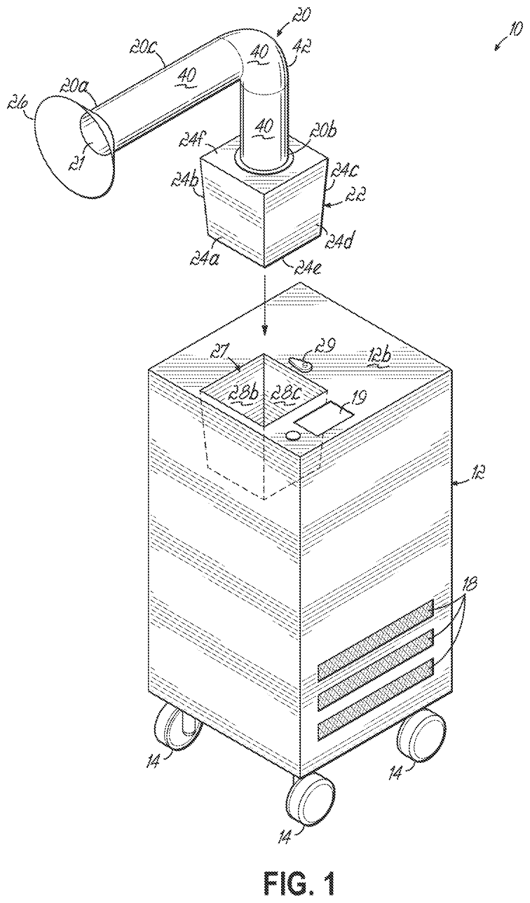

Referring now to FIGS. 1-10B, a surgical smoke evacuation system 10 is shown for evacuating smoke or vapor from a surgical site associated with a patient. As is well known, smoke and/or vapor may arise during a surgical procedure, for example, during use of a cautery instrument or the like. It is not uncommon that such smoke and vapor have toxic fumes or smoke that are dangerous to humans, such as personnel in an operating room or the patient.

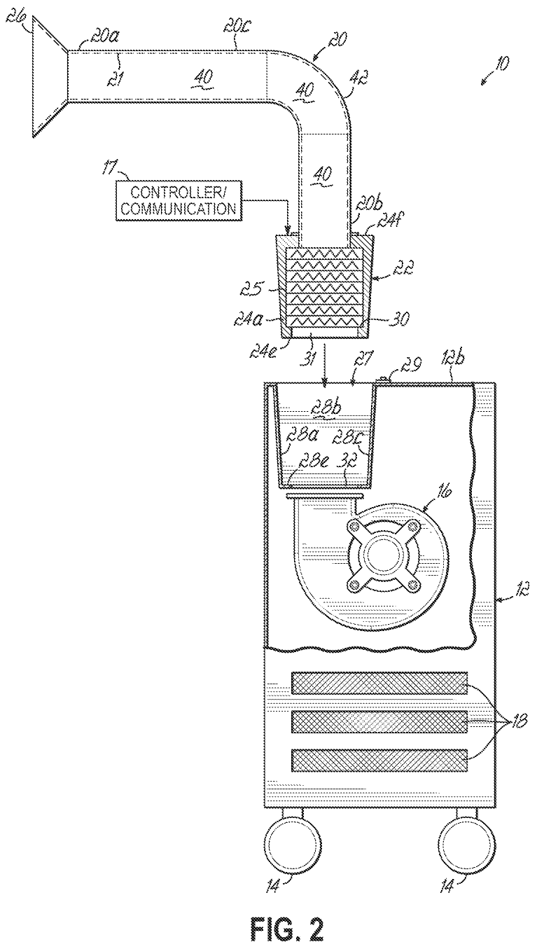

The surgical smoke evacuation system 10 comprises a vacuum generator 12 for generating a vacuum. The vacuum generator 12 is portable and comprises a plurality of wheels 14 for moving the vacuum generator 12 to a desired location. The vacuum generator 12 comprises a fan or blower 16 (FIG. 2) that generates a vacuum or negative pressure and airflow through the vacuum generator 12 and, ultimately, causes airflow through the exhaust 18. The vacuum generator 12 comprises a controller 19 for controlling an operation of the vacuum generator 12.

The vacuum generator 12 has a detachable, disposable and portable conformable tube member 20 that is received on and detachably mounted to the vacuum generator 12. Advantageously, the conformable tube member 20 is both disposable and detachable and is conveniently provided in a “drop in” mounting, as best illustrated in FIG. 1. The mounting features of the conformable tube member 20 will now be described.

The conformable tube member 20 has a first end 20a, a second end 20b and a tube member 20c that provides a substantially contiguous and open internal lumen or passageway 21 (FIG. 1) so that air can flow therethrough and be decontaminated in the manner described herein. Note that the first end 20a comprises a collection nozzle 26 that defines an opening or nozzle for the entry of surgical smoke or vapor generated during the surgical procedure. The collection nozzle 26 is press fit or detachably mounted to the tube member 20c by a conventional connection, such as a bayonet connection. When the controller 19 energizes the vacuum generator 12, a negative pressure or vacuum is generated at the nozzle 26 to suck contaminated air into and through the lumen or passageway 21.

The conformable tube member 20 also comprises a filter housing or support 22 that houses at least one or a plurality of filters 24 or other decontamination apparatus or means, which in the embodiment being described may comprise HEPA, ULPA, granular, chemical, catalytic, electrostatic, or similar means to perform or provide air decontamination of the air stream through the conformable tube member 20.

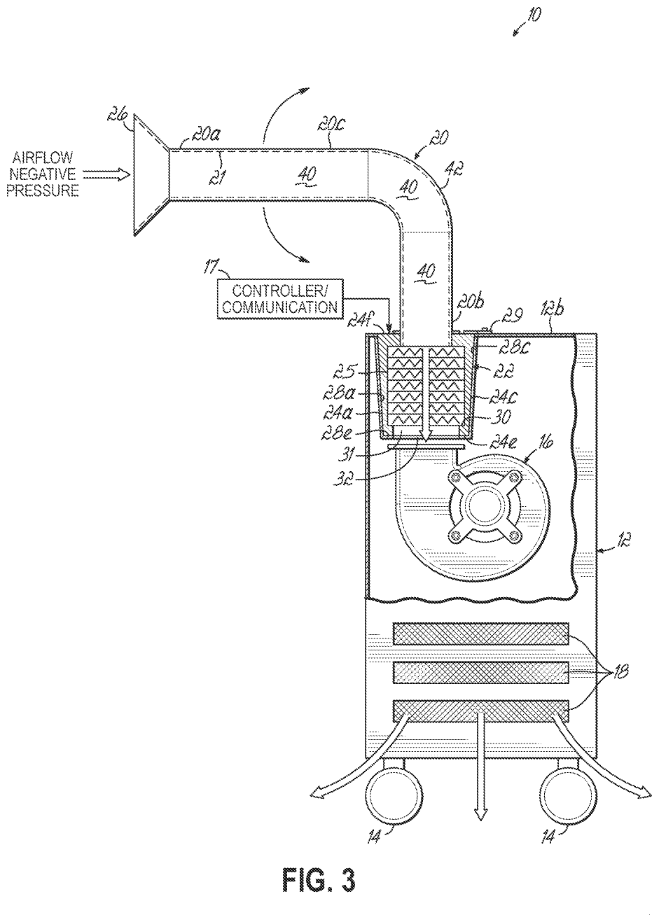

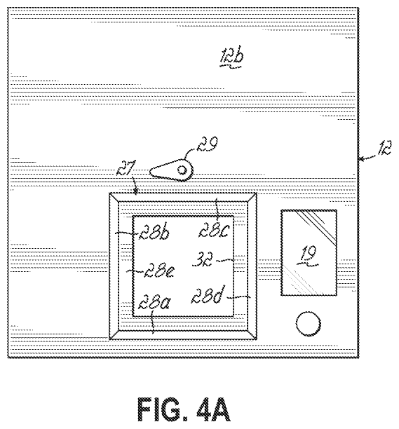

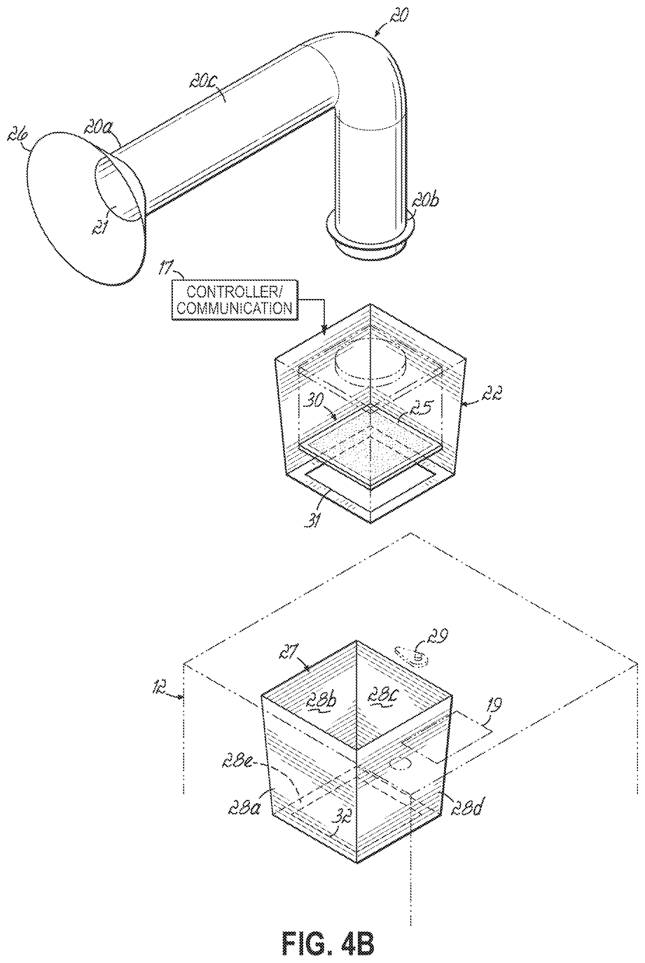

Referring now to FIG. 1, and as mentioned earlier herein, the vacuum generator 12 comprises the controller 19. As illustrated in FIG. 3, the filter housing or support 22 may comprise a controller/communication apparatus or means 17 (FIGS. 2 and 4B, for example). The controller/communication apparatus or means 17 is adapted to collect usage information and data such as the hours the filter has been used, contamination level, and other filter usage and contamination information that is communicated to the controller 19 of the generator and may also be used to monitor and control the decontamination efficiency of the surgical smoke evacuation system 10. One or more of features shown in the following U.S. Patents, which are owned by the Assignee of the present application, all of which are incorporated herein by reference and made a part hereof, may be used or adapted for use in the surgical smoke evacuation system 10: U.S. Pat. Nos. 9,433,693; 9,457,119; 9,764,054; 10,039,854; 10,702,435; 11,185,457; 11,285,237 and U.S. Patent Publication No. 2022/0031545.

Referring now to FIGS. 2 and 3, note that the filter housing or support 22 comprises a plurality of walls 24a-24f that define a housing and storage area 30 for storing at least one or a plurality of the filters 25 or other decontamination apparatus or means, such as HEPA, ULPA, granular, chemical, catalytic, electrostatic, or similar means to affect air decontamination.

FIG. 4A and FIG. 4B show a cavity 27 that is defined by a plurality of vacuum housing walls 28a-28d, and a generally rectangular support wall 28e for engaging and supporting the bottom wall 24e of the filter housing or support 22. FIG. 4B is a fragmentary and exploded view of a portion of the vacuum generator 12 illustrating how the conformable tube member 20 “drops in” or is detachably received or mounted in the cavity 27.

In FIG. 4B, the conformable tube member 20 is illustrated exploded from the filter housing or support 22 for ease of illustrating details hereof. Notice that the filter housing or support 22 is illustrated in FIG. 4B as being partially fragmented so that the surfaces that define the filter housing or support 22 may be shown and illustrated. In this regard, notice that the filter housing or support 22 comprises the walls 24a-24f that define a filter receiving area or aperture 30 for housing the at least one filter 25.

The filter housing or support 22 is adapted to support the at least one filter 25 so that it can filter the air as it passes through the conformable tube member 20. The at least one filter 25 is operatively coupled to the conformable tube member 20 and supported therein. It should be appreciated that the filter housing or support 22 could be all enveloping or not all enveloping. In other words, the filter housing or support 22 may be open and may comprise at least one or a plurality of fenestrations or openings 31, as illustrated in FIG. 2, so that the airflow flows through the at least one filter 25 and said filter housing or support 22. In the embodiments described herein, it is important to note that the filter housing or support 22 may comprise many different shapes as mentioned later herein. The filter housing or support 22 nests in the cavity 27 and the walls of the filter housing or support 22 engage and come into contact and are supported by the walls of the cavity 27.

Note that the walls 24a-24d cooperate with a bottom surface wall 24e and a top wall 24f to define the filter receiving area or aperture 30 therein. The bottom wall 24e is truncated or narrow so that it can support at least a portion of wall 24e of the filter housing or support 22 after it has been received in the cavity 27 in the manner described herein. The wall 24e defines an aperture 31 through which air may flow and pass into or through the cavity 27 and through an aperture 32 in the cavity 27.

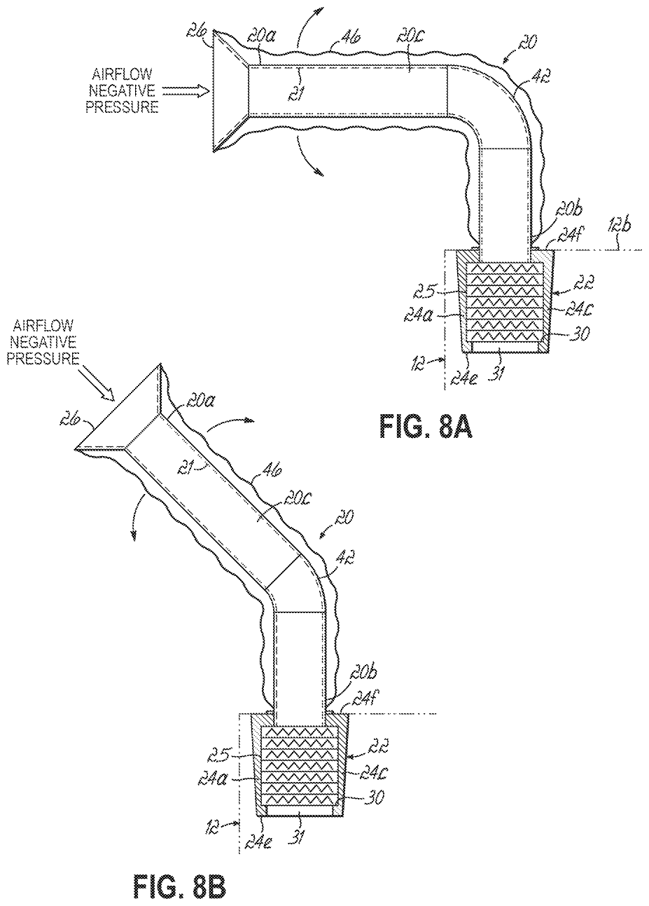

The bottom wall 28e of the cavity 27 cooperates with the walls 28a-28d of the cavity 27 for receiving the drop-in filter housing or support 22. In the illustration being described, the cavity 27 defined by the walls 28a-28e may comprise or define any predetermined shape that is adapted to permit the disposable conformable tube member 20 to be detachably mounted to the vacuum generator 12 by dropping the mating filter housing or support 22, which is complementarily shaped, into the cavity 27 as shown. Once the filter housing or support 22 has been received in the cavity 27, the conformable tube member 20 becomes fully supported and self-supported and can be manipulated by the user, as illustrated in FIGS. 8A-8B described later herein. Thus, the conformable tube member 20 has been received in the cavity 27, the nozzle 26 and conformable tube member 20 may be conformed, adapted or moved by a user so that the shape of the conformable tube member 20 can be changed and conformed and positioned to a desired location. In this regard, the nozzle 26 may be proximate to, but not have to intrude upon the surgical site. The conformable tube member 20 and the nozzle 26 may be placed in proximity to the surgical site and positioned outside a surgeon's line of sight or area of operation. Thus, the conformable tube member 20 is conformable and adaptable and can be reconfigured in response to the user positioning the nozzle 26 in a desired location, such as in proximity to the surgical site so that smoke and vapor may be captured. FIGS. 8A-8B illustrate the conformable tube member 20 being conformed or moved from the position in FIG. 8A to that shown in FIG. 8B.

The wall 24e defines the opening or aperture 30 that is in fluid communication with an aperture 32 that is defined by the bottom wall 28e of the vacuum generator 12 after the housing 22 has been placed or dropped into the cavity 27. The walls 24e and 28e engage so that not only are the openings or apertures 30 and 32 in fluid communication, but the filter housing or support 22 becomes fully supported and the conformable tube member 20 and/or nozzle 26 may be conformed to a desired location while the filter housing or support 22 remains captured in the cavity 27 and does not move. The filter housing or support 22 may be removed by the user when it is desired to remove the conformable tube member 20 and dispose of it, replace it entirely, or replace the at least one filter 25 in the filter housing or support 22. Although not shown, the at least one filter 25 may be situated in the filter housing or support 22 by inserting the at least one filter 25 through the aperture 30, or alternatively, at least one of the walls 24a-24f may be hinged to provide easy access to a filter receiving area or aperture 31 (FIG. 2) that is defined by filter housing walls 24a-24f, at least one or a plurality of locks or means for latching and securing the conformable tube member 20 to the vacuum generator 12 may be provided to lock the disposable and removable conformable tube member 20 the top wall 12b of the vacuum generator 12.

After the filter housing or support 22 has been dropped into the vacuum generator 12, as illustrated in FIGS. 2-4B, it may be locked into place. Accordingly, the surgical smoke evacuation system 10 may optionally include a lock, latch or other means 29 (FIG. 3), such as a mechanical or magnetic latch or lock, that locks the filter housing or support 22 to the vacuum generator 12 into position with the cavity 27.

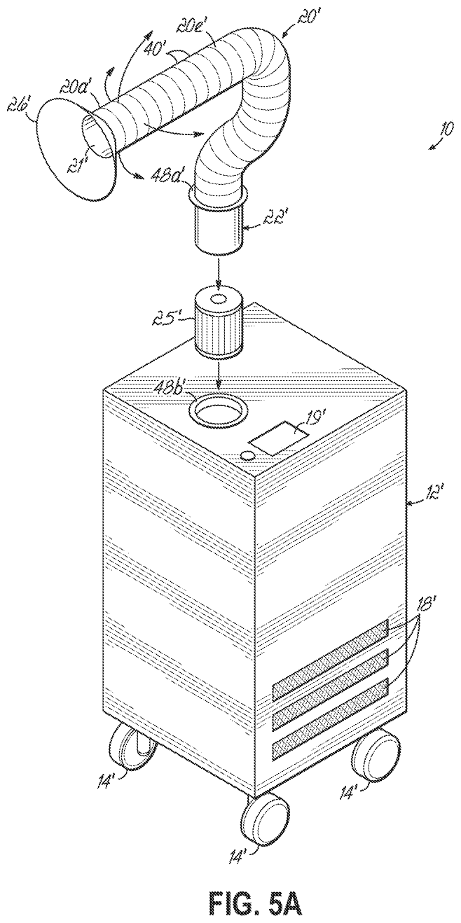

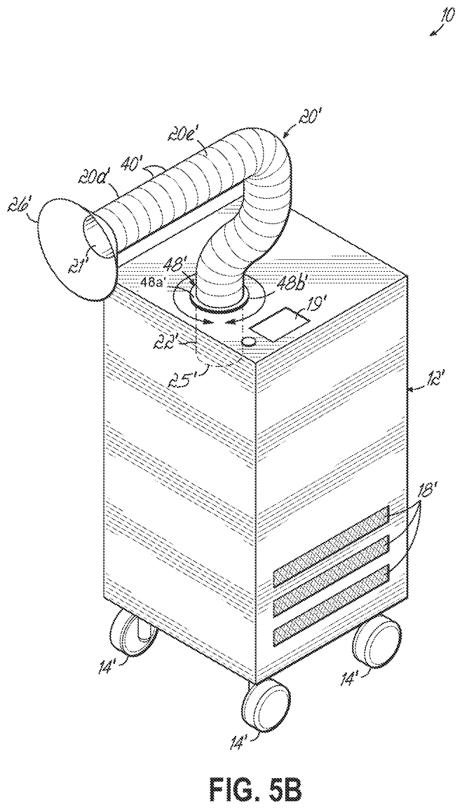

During use, the nozzle 26 is positioned and conformed to a desired location by the user, such as a medical technician, nurse or doctor, who positions the nozzle 26 in proximity to the surgical site, but not in obstruction of the surgical procedure. FIGS. 5A-7B illustrate several other embodiments of the surgical smoke evacuation system 10 and conformable tube member 20. In these embodiments, the same or similar parts are identified with the same part numbers, except that one or more prime marks (“′”,“″”, “″′”) have been added to the part numbers for the different embodiments of FIGS. 5A-7B.

It should be appreciated that in all embodiments, the conformable tube members 20, 20′, 20″ or 20′″ are segmented and made up of at least one or a plurality of segments 40, 40′, 40″ or 40″ that enable the nozzle 26, 26′, 26″ or 26″ to be conformed to any desired shape or any desired position while having memory and retaining conformity.

FIGS. 5A-5B illustrate the nozzle 26′ and conformable tube member 20′ having the plurality of segments 40′ that are conformed to a generally sideways U-shape as illustrated. FIG. 5A illustrates the conformable tube member 20′ exploded from the vacuum generator 12′. Notice that the second end 20b′ (FIG. 5B) comprises the at least one filter 25′ which in this embodiment is generally cylindrical and situated on the second end 20b′ of the conformable tube member 20′. As illustrated in FIG. 5B, the at least one filter 25′ and the conformable tube member 20′ are dropped in the vacuum generator 12′ as illustrated in FIG. 5B. A sleeve or mount 48′ may be used to fix the conformable tube member 20′ to the vacuum generator 12′. The optional sleeve or mount 48′ may comprise a first collar 48a′ and a second collar 48b′ that mate and secure the conformable tube member 20′ to the vacuum generator 12′. In this regard, the collars 48a′ and 48b′ may have fasteners, such as screws, a bayonet connection, a threaded connection or the like.

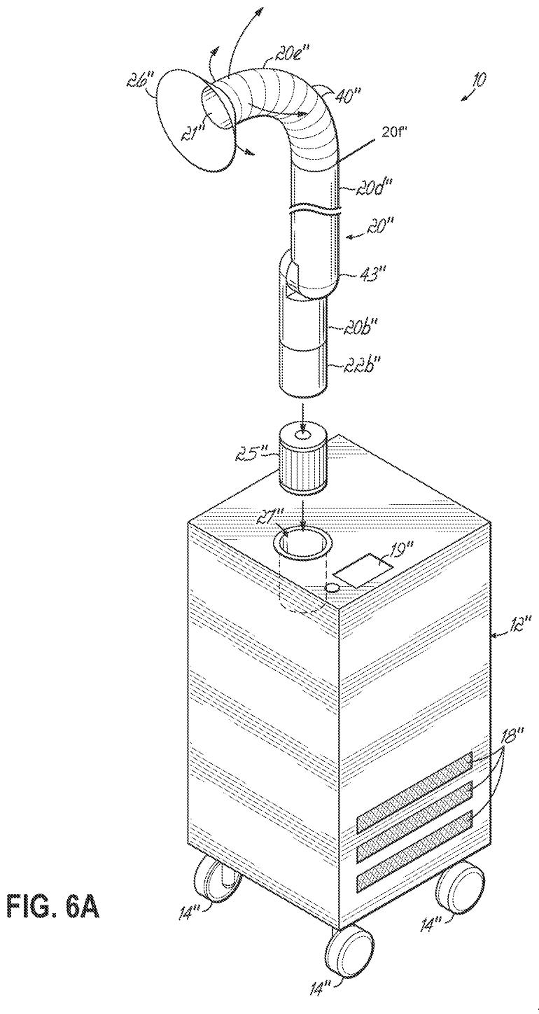

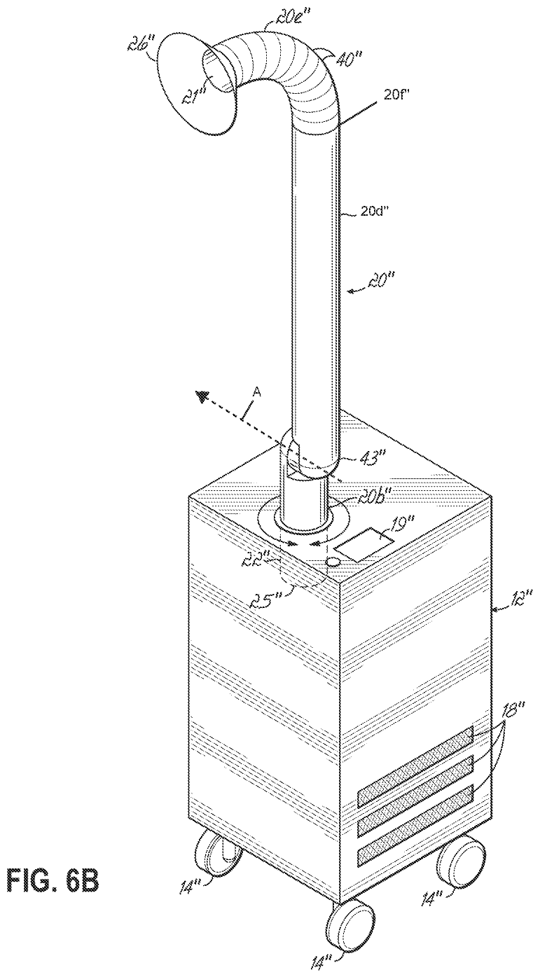

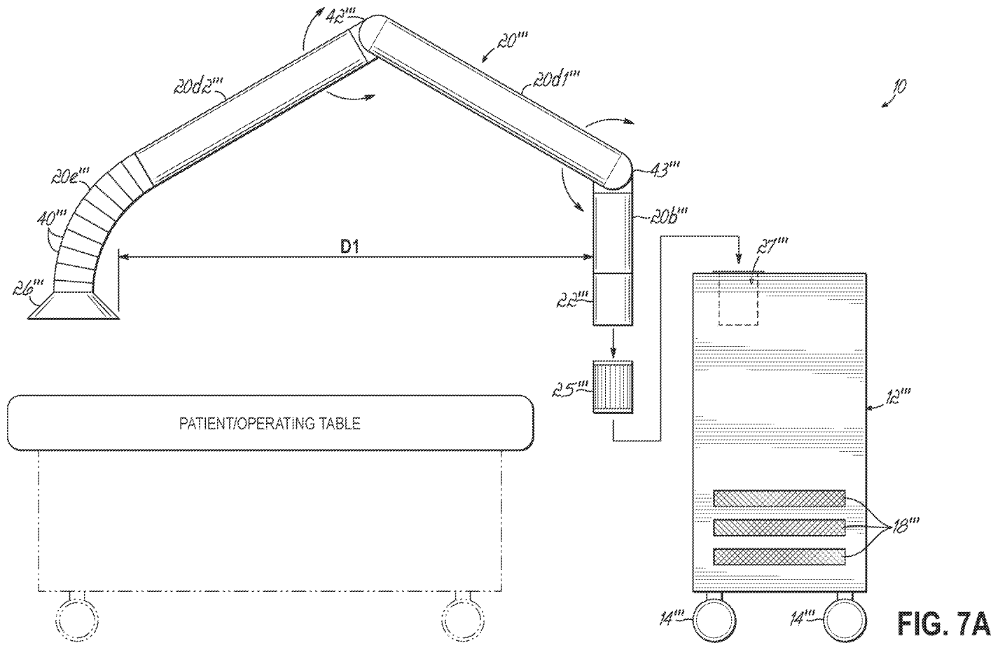

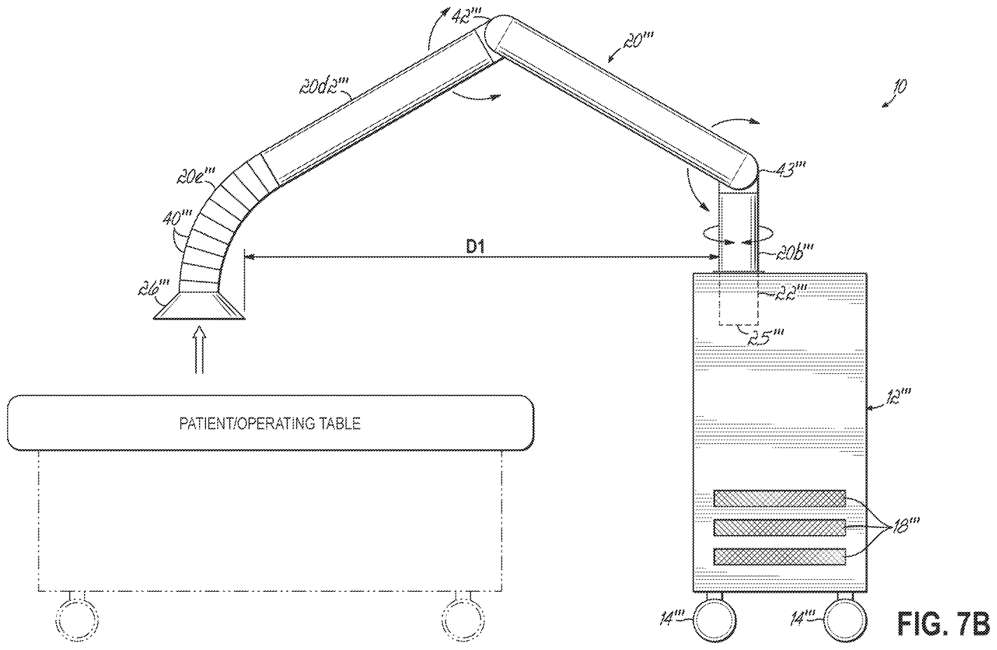

Notice that in FIGS. 6A-6B that the conformable tube member 20″ may have an elongated or intermediate portion 20d″ between the nozzle 26″ and the second end 20b″ coupling a conformable end portion 20e″ having the nozzle 26″ and a base or drop-in end 20f″. Alternatively, a plurality of elongated tubular intermediate portions and 20d2″ (FIGS. 7A-7B) is integrally formed with or coupled to the nozzle 26″ as illustrated in FIG. 6B.

Similar to the embodiment of FIGS. 5A and 5B, the embodiment of FIGS. 6A and 6B also comprise the conformable tube member 20″ as shown. In this embodiment, the surgical smoke evacuation system 10″ may also comprise an elongated tubular intermediate portion 20d″. The elongated tubular intermediate portion is coupled to the conformable tube member 20″ and comprises the second end having the at least one filter 25″ situated, housed or supported therein. Notice in FIG. 6B that the second end 20b″ has been dropped into and nested into the vacuum generator 12″. In the embodiment of FIGS. 6A-7B, it should be understood that the generally cylindrical second end 20b″, 20b″ enables the conformable tube member 20″, to be rotated about an axis of the second end 20b″, 20b′″ as illustrated. A sealed pivot joint 43″ connects the second end 20b″ to the elongated tubular intermediate portion 20d″ and can pivot about the axis A in FIG. 6B.

Advantageously, the pivoting movement of the elongated tubular intermediate portion 20d″ enables the user to manipulate and move the first end 20a″ or nozzle 26″ to the desired location, such as in proximity to a surgical site easily and conveniently.

One or more intermediate segments or elongated intermediate portions 20d″, 20d1″, 20d1″, 20d2″ or 20d2′″ (FIGS. 6A and 7A) may be coupled to and provide additional length to the conformable tube member 20, 20′, 20″ or 20″. Notice in the embodiments of FIGS. 6A-6B that the second end 20b″ has been inserted into a mating cavity 27″ that retains and supports the conformable tube member 20″ on the vacuum generator 12″ and in the position shown in FIG. 6B. FIG. 6A illustrates the second end 20b″ before it has been inserted into the cavity 27″. Once the second end 20b″ is inserted into the cavity 27″, the conformable tube member 20″ is self-supported so that the conformable tube member 20″ can be conformed or moved and the position of the nozzle 26″ changed so that the conformable tube member 20″ is both self-supporting and also conformational and can be changed by the user so that the nozzle 26″ may be positioned in a desired location. The conformable tube member 20″ has memory because of the frictional engagement between and among the various segments 40″ in order to retain the conformable tube member 20″ into the conformed position, such as the position shown in FIGS. 1-7B. In the illustration, the filter housing or support 22″ is defined by the inside portions of the second end 20b″ that is sized and adapted to receive the at least one filter 25″.

FIGS. 7A-7B illustrate another embodiment of the conformable tube member 20″ having the multiple intermediate portions, such as 20d1′″ and 20d2′″ mentioned earlier. Notice in FIG. 7A that the end of the nozzle 26″ has been conformed and positioned in proximate relationship to a patient and a surgical site. When the nozzle 26″ has been conformed as illustrated, the conformable tube member 20″ retains its conformation so that user intervention is not necessary. The conformable tube member 20″ will retain its shape after conformation by the user. Notice in the illustration in FIGS. 7A-7B, that a very large distance D1 between the vacuum generator 12″ and the nozzle 26″ can span any predetermined distance by selecting intermediate portions 20d1′″ and 20d2′″ of desired length. In this regard, the conformable tube member 20′″ may be provided with intermediate portions 20d1″ and 20d2′″ that are interchangeable in order to change an overall length D1 (FIG. 7A) of the conformable tube member 20′″. Thus, more or fewer sections may be added to the conformable tube member 20″ to make it longer or shorter if desired.

FIG. 7A illustrates the conformable tube member 20″ having a plurality of intermediate sections 20d1″ and 20d2′″ that are coupled by a first sealed pivot joint 42″ and the second sealed pivot joint 43″ which connects the intermediate portion 20d1′″ to the second end 20b″ which houses or supports the at least one filter 25″. The second end 20b″ is inserted and mounted to the vacuum generator 12″ as illustrated in FIG. 7B. Once mounted, the plurality of intermediate portions 20d1′″ and 20d2′″ and the conformable tube member 20′″ may be manipulated and/or pivoted so that the user can position the nozzle 26″ in a desired location such as in proximity to a patient table (FIG. 7B) where a surgical procedure is being performed.

Advantageously, in the embodiments of FIGS. 5A-7B, the conformable tube member 20′, 20″, 20′″ may be manipulated, rotated, pivoted and moved such that the nozzle 26′, 26″, 26″ is situated in a desired location. In each of these embodiments, notice that the at least one filter 25′, 25″, 25″ is in the second end 20b′, 20b″, 20b′″ of the conformable tube member 20′, 20″, 20′″ and then dropped in or nested into the cavity 27′, 27″, 27″ of the vacuum generator 12′, 12″, 12′″ as shown. In the illustrations of FIGS. 1-4B, 6A-6B, 7A-7B, 8A-8B, the conformable tube member 20″ has sealed pivot joints 42″ that not only permit the portions 20d1″ and 20d2′″ to pivot relative to each other and relative to the vacuum generator 12″ while allowing airflow to flow into the nozzle 26″, through the internal lumen 21′″ of the conformable tube member 20″, through the pivot joints 42″ and into the second end 20b′″ wherein the at least one filter 25″ is housed. In this regard, notice that the second end 20b′″ is generally circular whereas the end in the embodiment of FIGS. 1-4B is pyramidally shaped in contrast.

It should be understood that the ends 20b, 20b′, 20b″ or 20b″ of the conformable tube member 20, 20′, 20″ or 20″ comprise the filter housing or support 22, 22′, 22″ or 22″ and may comprise any predetermined shape that is adapted and suited to support and retain the conformable tube member 20″ onto the vacuum generator 12″, while permitting the nozzle 26″ to be conformed to a desired position. The conformable tube member 20′″ has the continuous and contiguous internal lumen or passageway 21″ that permits airflow to pass through the entire length of the conformable tube member 20′″.

As mentioned earlier, the conformable tube member 20″ may comprise a plurality of segments 40″ that are coupled together as shown and permit the nozzle 26″ to be conformed to a desired position, with the segments 40′″ cooperating to retain the conformable tube member 20′″ into the conformed position performed by a user. Notice in the embodiments of FIGS. 6A-7B, that the second ends 20b″ and 20b′″, respectively, are generally cylindrical and drop into a mating female cavity or generally cylindrical complimentary shaped cavity 27″ so that after the second end 20b″ is situated in the cavity 27″, the conformable tube member 20′″ is self-supported and can be conformed by the user as described herein.

Advantageously, it should be appreciated that the conformable tube member 20, 20′, 20″ and 20′″ is self-supporting and conformable and detachable from the vacuum generator 12, 12′, 12″ and 12′″. It can be re-shaped by the user while remaining in substantially the shape that the user selects. This allows for placement of the nozzle 26, 26′, 26″ or 26″ in proximity to a desired location, such as a patient surgical site without requiring direct patient contact. It is also disposable and preferably replaced after a predetermined time.

Thus, it should be understood that the conformable tube member 20 is detachable and replaceable as a single unit with the filter housing or support 22 comprising a substantially integrated assembly with the at least one filter 25 filtering the air as it passes through the conformable tube member 20. In a preferred embodiment, each of the conformable tube members 20, 20′, 20″ and 20′″ illustrated in FIGS. 1-7B are segmented as described herein and conformable as described and shown herein. It is contemplated that a plurality of different-shaped conformable tube members 20, 20′, 20″ and 20′″ may be provided for use with the vacuum generator 12, 12′, 12″ and 12′″. For example, some portions of the conformable tube member 20, 20′, 20″ and 20″ may be elongated, curved and the like. Some portions of the conformable tube member 20, 20′, and 20′″ may be elongated or the diameter of the conformable tube member 20, 20′, and 20′″ may be smaller or larger if desired.

Moreover, the nozzle 26 may comprise a predetermined shape selected by the user if desired. In this regard, the user selects the conformable tube member 20 that is desired. The nozzle 26 may be integrally formed onto the first end 20a of the conformable tube member 20 or it may be detachably secured thereto by, for example, a press-fit, bayonet coupling, threaded coupling or the like. The shape of the end of the nozzle 26 may also be changed to comprise an elongated rectangular shape analogous to a vacuum cleaner nozzle attachment. FIGS. 10A and 10B illustrate two exemplary embodiments of two tapering nozzles 26, one with a generally cylindrical inlet 26a, one with a generally rectangular inlet 26b, respectively. Obviously, with the narrowing of the inlets 26a and 26b of nozzle 26 into the conformable tube member 20, a negative pressure at the nozzle 26 is increased compared to the airflow at the second end 20b.

As with the prior embodiments, the conformable tube member 20 comprises the filter housing or support 22 that is dropped into the cavity 27 of the vacuum generator 12 as illustrated in FIGS. 1-4B, for example, and as described herein. In the illustration of FIGS. 1-4B, the filter housing or support 22 is pyramidal and the cavity walls 28a-28d are formed to provide the mating female cavity 27 that has a complementary shape to the filter housing or support 22. After the filter housing or support 22 is dropped into the vacuum generator 12, the conformable tube member 20 becomes secured to the vacuum generator so that the nozzle 26 can be conformed to a desired position by a user. In the illustration being described, the filter housing or support 22 comprises at least one or a plurality of filters or filtration means. In the illustration being described, the at least one filter 25 may comprise HEPA, ULPA, granular, chemical, catalytic, electrostatic, or similar means to affect air decontamination.

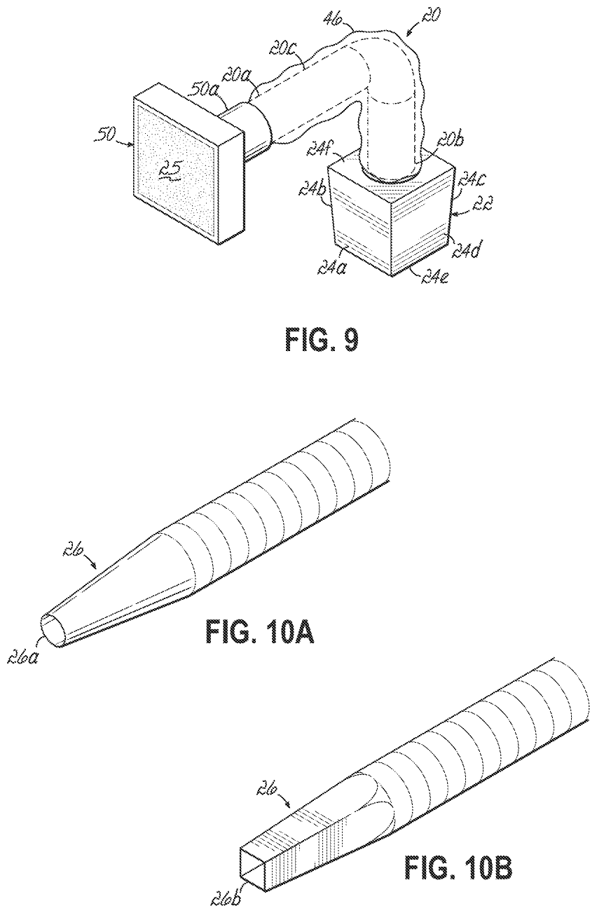

As illustrated in FIGS. 8A-8B and FIG. 9, the conformable tube member 20 may comprise a sterile, flexible external drape or sheath 46 for protecting users from contamination. The sheath may be detachably mounted to or integral from the nozzle 26 to the second end 20b if desired to protect the conformable tube member 20 and to provide a sterile drape or sheath 46 that the user can grip when manipulating the position of the nozzle 26. It should be understood that the movable drape or sheath 46 may be disposable and separately applied to the conformable tube member 20 or it may be integrally formed to the conformable tube member 20. In this regard, it is contemplated that the conformable tube member 20 and filter housing or support 22 are disposable so that after a single use or a predetermined number of uses, the conformable tube member 20 can be removed from the vacuum generator 12 and simply discarded.

FIGS. 8A-8B illustrate a conformable tube member 20 having a single joint 42, illustrating the movement of conformable tube member 20. In the example, FIG. 8A illustrates a sterile sheath or drape over conformable tube member 20. FIG. 8B illustrates the conformable tube member 20 after the nozzle 26 has been moved to the position shown in FIG. 8B. Notice that once moved from the position in FIG. 8A to the position in FIG. 8B, the conformable tube member 20 remains in that position until adjusted by a user. As mentioned earlier, FIGS. 7A-7B illustrate a conformable tube member 20 with multiple joints 42, 43.

FIG. 9 illustrates yet another embodiment showing the at least one filter 25 at the first end 20a of the conformable tube member 20. In the embodiment of FIG. 9, a filter nozzle 50 comprises the at least one filter 25 therein. The end 50a of the filter nozzle 50 is press-fit or slidably mounted onto the first end 20a of the conformable tube member 20. In the illustration being described, additional filter 25 may also be used inside the filter housing or support 22 or the conformable tube member 20 if desired. Alternatively, the filter housing or support 22 may be provided so that there is no filter 25 resident therein.

Additional Considerations
1. While the conformable tube member 20 and the passageway or lumen 21 that extends from the nozzle 26 through the second end 20b have been shown as being generally cylindrical, it should be understood that the conformable tube member 20 may comprise different cross-sectional tubular shapes, such as a polygonal shape. Thus, while the embodiments shown and described herein illustrate the conformable tube member 20 as being generally cylindrical, it could take other types of polygonal or tubular shapes, like a rectangle, square or the like.
2. While the embodiments illustrated show a generally constant configuration or shape from the inlet end 20a to the second end 20b, it should be understood that the conformable tube member 20 may have different shapes along its length as well.
3. An important feature of the embodiment being described is that the conformable tube member 20 may comprise the lock or latch or means 29 (FIG. 3) for locking or securing the conformable tube member 20 to the vacuum generator 12. Alternatively, the second end 20b may comprise a mount 48 (FIG. 5B, for example). The mount 48 may comprise a male and female portion, one of which is mounted on the vacuum generator 12 and the other integrally formed in the second end 20b. The two collars 48a and 48b cooperate and secure the conformable tube member 20 to the vacuum generator 12 as illustrated in FIG. 5B in the event there is no “drop-in” cavity, like cavity 27. Thus, while the embodiments shown in FIGS. 1, 6A-6B and 7A-7B illustrate the second end 20b of the conformable tube member 20 being simply dropped into a mating cavity in the vacuum generator 12, it is also contemplated, as illustrated in FIG. 5B, that the second end 20b of the conformable tube member 20 be detachably affixed or secured to the vacuum generator 12 in other ways as well. In this regard, the mount 48 may comprise bayonet connection, mating snap-fit connection, press-fit connection, threaded connection or other suitable connection adapted to retain the conformable tube member 20 on the vacuum generator 12 while permitting the nozzle 26 to be conformed and moved by the user as described herein.
4. The components of the surgical smoke evacuation system 10, including the conformable tube member 20 and the vacuum generator 12, may be obtained from Aerobiotix, LLC, located in Miamisburg, Ohio.
5. Advantageously, the self-supporting conformable tube member 20 provides means and apparatus for providing a disposable and sterile conformable tube member 20 that may be easily removed from the vacuum generator 12 and conveniently disposed of and replaced with a replacement conformable tube member 20 with or without the sterile sheath or drape 46. As mentioned earlier herein, the conformable tube member 20 may comprise the same general shape and the conformable tube member 20 may be selected and mounted onto the vacuum generator 12 as described herein. The replacement conformable tube member 20 may be the same or it may be different, or it may have the same or a different nozzle 26 shape. Thus, it is important to note that the conformable tube member 20 is not only detachable and disposable, but it can be easily replaced with the same, similar, or even a different conformable tube member 20.
6. Advantageously, the system may comprise the controller/communication apparatus or means 17 that facilitate controlling the amount of decontamination filtered by the at least one filter 25 and to control the efficiency of its operation.

Advantageously, another embodiment of this invention, including all embodiments shown and described herein, could be used alone or together and/or in combination with one or more of the features covered by one or more of the claims set forth herein, including but not limited to one or more of the features or steps mentioned in the Summary of the Invention and the claims.

While the system, apparatus and method herein described constitute preferred embodiments of this invention, it is to be understood that the invention is not limited to this precise system, apparatus and method, and that changes may be made therein without departing from the scope of the invention which is defined in the appended claims.

1. A surgical smoke evacuation system for evacuating smoke or vapor from a surgical site associated with a patient, said surgical smoke evacuation system comprising:
a vacuum generator for generating a vacuum;
a conformable tube member having a first end having an opening or nozzle for entry or passage of surgical smoke or vapor generated during a surgical procedure, said conformable tube member also having a second end having a filter housing adapted to receive and to treat said surgical smoke or vapor passing between said first end and said second end and through said conformable tube member; and
at least one of said first end or said second end being removably mounted to said vacuum generator such that said opposite end may be positioned in proximity to said surgical site.

2. The surgical smoke evacuation system as recited in claim 1 wherein said vacuum generator comprises a vacuum generator housing having a filter housing or support receiving area adapted and sized to mate with and receive said filter housing or support.

3. The surgical smoke evacuation system as recited in claim 2 wherein said filter housing or support receiving area comprises a vacuum generator predetermined shape for removably receiving and supporting said filter housing or support such that said conformable tube member can be conformed or moved to enable said first end to be moved or conformed to a proximity at or near the surgical site.

4. The surgical smoke evacuation system as recited in claim 3 wherein said filter housing or support has a filter housing or support predetermined shape that generally complements said vacuum generator predetermined shape so that said filter housing or support can be received and stored therein so that said conformable tube member can be detachably mounted to said vacuum generator.

5. The surgical smoke evacuation system as recited in claim 3 wherein said vacuum generator predetermined shape defines a filter housing or support receiving area for receiving said filter housing or support, said filter housing or support having a predetermined filter housing or support shape that generally complements a shape of said filter housing or support so that after said filter housing or support is received in said filter housing or support receiving area, said conformable tube member is in fluid communication with said vacuum generator so that said vacuum generator can cause a negative pressure through said conformable tube member so that said smoke and vapor may be collected through said first end after said first end has been placed in proximity to said surgical site by a user.

6. The surgical smoke evacuation system as recited in claim 5 wherein said at least one of said vacuum generator or said conformable tube member comprise at least one securing means or lock for securing or locking, respectively, filter housing or support onto said vacuum generator.

7. The surgical smoke evacuation system as recited in claim 5 wherein said vacuum generator predetermined shape is sized and dimensioned to receive said filter housing or support such that it is secure and stabilized during movement of said conformable tube member.

8. The surgical smoke evacuation system as recited in claim 7 wherein said vacuum generator comprises a vacuum generator housing having a filter housing or support receiving area for receiving said filter housing or support.

9. The surgical smoke evacuation system as recited in claim 5 wherein said vacuum generator predetermined shape is at least one of a cylinder, polyhedron or is pyramidal.

10. The surgical smoke evacuation system as recited in claim 1 wherein said conformable tube member is reshapable and conformable by the user so that at least one of said first end or said second end can be situated in proximity to said surgical site, said conformable tube member having memory and being self-supporting so that it remains in position after the user has positioned said first end.

11. The surgical smoke evacuation system as recited in claim 1 wherein said filter housing or support has a filter housing or support predetermined shape that generally complements a vacuum generator predetermined shape so that said filter housing or support can be received therein so that said conformable tube member can be detachably mounted to said vacuum generator.

12. The surgical smoke evacuation system as recited in claim 1 wherein said vacuum generator has a vacuum generator controller, said filter housing or support comprises communication means or a filter housing or support control for communicating data to and/or from said vacuum generator controller regarding a state or condition data of said at least one filter, said state or condition data comprising data relating to at least one of usage time, filter expiration or usage factors.

13. The surgical smoke evacuation system as recited in claim 12 wherein said filter housing or support comprises an air sensor coupled to said vacuum generator controller for sensing a plurality of parameters, said plurality of parameters comprising carbon dioxide, volatile organic compound, particulate, pressure, temperature, humidity and/or other physical parameters.

14. The surgical smoke evacuation system as recited in claim 1 wherein said conformable tube member further comprises a sterile flexible drape or sheath situated on said conformable tube member.

15. The surgical smoke evacuation system as recited in claim 1 wherein said conformable tube member further comprises a sterile flexible drape or sheath situated on said conformable tube member, said sterile flexible drape or sheath being removably mounted on said conformable tube member so that it can be removed and discarded and replaced with another one of said sterile flexible drape or sheath.

16. The surgical smoke evacuation system as recited in claim 1 wherein said filter housing or support may comprise a predetermined shape that is at least one of a cylinder, polyhedron or is pyramidal.

17. The surgical smoke evacuation system as recited in claim 1 wherein said first end is an inlet end, said filter housing or support being situated on said inlet end.

18. The surgical smoke evacuation system as recited in claim 1 wherein said conformable tube member can be reversibly re-shaped by the user and will substantially remain in said shape, allowing for placement proximate to a surgical site, without requiring direct patient contact, a self-supporting tube mechanism or mechanisms integral to and/or acting upon said tube resulting in self-supporting conformational change of said tube by the user, while maintaining a substantially contiguous internal lumen.

19. The surgical smoke evacuation system as recited in claim 17 wherein said inlet end comprises a nozzle.

20. The surgical smoke evacuation system as recited in claim 18 wherein said self-supporting tube mechanism and said filter housing or support comprise a substantially integrated assembly which can be handled or replaced a single unit, said tube and said housing further comprising a contiguous air flow channel, said channel comprising a lumen being substantially air and water tight along its length from said first tube opening to said filter housing or support and through an airflow aperture.

21. The surgical smoke evacuation system as recited in claim 1 wherein said filter housing or support comprises an air filtration means, including HEPA, ULPA, granular, chemical, catalytic, electrostatic, or similar means to affect air decontamination.

22. The surgical smoke evacuation system as recited in claim 1 wherein said vacuum incorporates an air sensing means, said air sensing means comprising modalities including carbon dioxide, volatile organic compound, particulate, pressure, temperature, humidity and/or other physical parameters, said parameters feeding back into said air sensing means for display, memory, or transmission.

23. The surgical smoke evacuation system as recited in claim 1 wherein said conformable tube member comprises a sterile flexible external drape or sheath.

24. The surgical smoke evacuation system as recited in claim 1 wherein said conformable tube member is manufactured from a re-sterilizable metallic or polymeric material, such as stainless steels, polyethylenes, polysulphones, silicones, and/or acetals for autoclave sterilization.

25. The surgical smoke evacuation system as recited in claim 1 wherein said conformable tube member comprises a detachable distal nozzle, which can be sized or shaped to anatomical requirements of a specific surgical procedure.

26. A disposable surgical smoke evacuation tube for use with a surgical smoke evacuation system having a vacuum generator, said disposable surgical smoke evacuation tube comprising:
a conformable tubular body member having a first end and a second end;
at least one filter housing or support for housing at least one filter, said at least one filter housing or support being coupled to and in fluid communication with said conformable tubular body member;
said conformable tubular body member being adapted and shaped to be removably secured or mounted to a vacuum generator receiving area of said vacuum generator such that it is stable so that a user can position said filter housing or support at said receiving area so that said vacuum generator can generate a vacuum through said conformable tubular body member so that at least one of said first or second ends can be positioned in proximity to a surgical site to evacuate smoke and vapor from said surgical site associated with a patient.

27. The disposable surgical smoke evacuation tube as recited in claim 26 wherein said conformable tubular body member has memory so that at least one of said first or second end can be positioned placed in proximity to said surgical site and remain conformed in a desired location.

28. The disposable surgical smoke evacuation tube as recited in claim 26 wherein said at least one filter housing or support is located on at least one of said first or second ends and in fluid communication with said tubular body member.

29. The disposable surgical smoke evacuation tube as recited in claim 28 wherein said at least one filter housing or support is situated on an outlet end of said conformable tubular body member, said at least one filter housing or support having a predetermined filter housing or support shape, the vacuum generator having a vacuum generator receiving area that is sized, shaped and adapted to generally complement the shape of said predetermined filter housing or support shape so that said at least one filter housing or support can be situated in said vacuum generator receiving area and coupled to the vacuum generator so that an inlet end of said conformable tubular body member may be positioned at or near the surgical site.

30. The disposable surgical smoke evacuation tube as recited in claim 29 wherein said vacuum generator receiving area is defined by a plurality of walls sized and dimensioned and configured to mate with and receive said filter housing or support such that it is secure and stabilized during movement of said first end.

31. The disposable surgical smoke evacuation tube as recited in claim 30 wherein said predetermined filter housing or support shape is at least partially cylindrical, polyhedron or pyramidal in shape.

32. The disposable surgical smoke evacuation tube as recited in claim 31 wherein said predetermined filter housing or support shape and said vacuum generator receiving area are both at least partially cylindrical, polyhedron or pyramidal and complementary in the shape so that said at least one filter housing or support can be received and secured in said receiving area, thereby coupling said tubular body member to the vacuum generator.

33. The disposable surgical smoke evacuation tube as recited in claim 26 wherein said conformable tubular body member comprises a sterile protective cover or sheath.

34. The disposable surgical smoke evacuation tube as recited in claim 33 wherein said sterile protective cover or sheath is both removeable and disposable.

35. The disposable surgical smoke evacuation tube as recited in claim 26 wherein at least one of the vacuum generator or said tubular body member comprises at least one securing means or lock for securing or locking, respectively, at least one filter housing or support to the vacuum generator.

36. The disposable surgical smoke evacuation tube as recited in claim 26 wherein said conformable tubular body member is re-shapable and conformable by the user so that an inlet end can be situated in proximity to a surgical site, said conformable tubular body member having memory and being self-supporting so that it remains in position after the user has positioned or conformed said first end in proximity to said surgical site.

37. The disposable surgical smoke evacuation tube as recited in claim 36 wherein said at least one filter housing or support is situated on and in fluid communication with said first end.

38. The disposable surgical smoke evacuation tube as recited in claim 26 wherein the vacuum generator comprises a vacuum generator controller, said filter housing or support comprising a data collector or collection means for collecting usage data regarding the filter and for communicating said usage data to and/or from said vacuum generator controller regarding a state or condition data of said at least one filter, said state or condition data comprising data relating to at least one of usage time, filter expiration or usage factors.

39. The disposable surgical smoke evacuation tube of claim 26, wherein said conformable tubular body member can be reversibly re-shaped by the user and will substantially remain in said shape, allowing for placement proximate to a surgical site, without requiring direct patient contact, a self-supporting tube mechanism or mechanisms integral to and/or acting upon said tube resulting in self-supporting conformational change of said tube by the user, while maintaining a substantially contiguous internal lumen.

40. The disposable surgical smoke evacuation tube of claim 26, wherein said surgical smoke evacuation system comprises a plurality of conformable tubes having different shapes, at least one of which is selected for different anatomical needs or situations.

41. The disposable surgical smoke evacuation tube of claim 26, wherein said conformable tubular body member can be reversibly re-shaped by the user and will substantially remain in said shape, allowing for placement proximate to a surgical site, without requiring direct patient contact, a self-supporting tube mechanism or mechanisms integral to and/or acting upon said tube resulting in self-supporting conformational change of said tube by the user, while maintaining a substantially contiguous internal lumen.

42. The disposable surgical smoke evacuation tube as recited in claim 26, wherein an inlet end comprises a nozzle.

43. A surgical smoke evacuation system comprising a conformable self-supporting tube with a first tube end comprising an opening or nozzle for the entry of surgical smoke, said first tube end further comprising filtration means or filter, said filtration means or filter being in an operative relationship with said opening or nozzle, said self-supporting tube comprising a second tube end dimensioned for attachment to at least one of a vacuum and or filtration means.

44. The surgical smoke evacuation system as recited in claim 43, wherein said filtration means or filter comprises filtration media having at least one of a filter, a holder, an enclosure housing, textile, granules, or energy-based smoke treatment means or ultraviolet light (UV) treatment.

45. The surgical smoke evacuation system as recited in claim 43, wherein said opening or nozzle comprises an inlet nozzle dimensioned to be removable and/or replaceable from said first tube end and further comprising a mechanical securing means comprising at least one of a clip, fastener, magnet, compression, friction, nesting or similar means.

46. The surgical smoke evacuation system as recited in claim 43 wherein said first end is an inlet end, said filter housing or support being situated on said inlet end.

47. The surgical smoke evacuation system as recited in claim 43 wherein said conformable tube can be reversibly re-shaped by the user and will substantially remain in said shape, allowing for placement proximate to a surgical site, without requiring direct patient contact, a self-supporting tube mechanism or mechanisms integral to and/or acting upon said tube resulting in self-supporting conformational change of said tube by the user, while maintaining a substantially contiguous internal lumen.

48. The surgical smoke evacuation system as recited in claim 46, wherein said inlet end comprises a nozzle.

49. The disposable surgical smoke evacuation tube as recited in claim 48 wherein a predetermined filter housing or support shape and a vacuum generator receiving area are both at least partially cylindrical, polyhedron or pyramidal and complementary in the shape so that said at least one filter housing or support can be received and secured in said vacuum generator receiving area, thereby coupling said conformable self-supporting tube to the vacuum generator.

50. The disposable surgical smoke evacuation tube as recited in claim 43 wherein said conformable self-supporting tube comprises a sterile protective cover or sheath.

51. The disposable surgical smoke evacuation tube as recited in claim 50 wherein said sterile protective cover or sheath is both removeable and disposable.

52. The disposable surgical smoke evacuation tube as recited in claim 43 wherein at least one of the vacuum generator or said conformable self-supporting tube comprises at least one securing means or lock for securing or locking, respectively, at least one filter housing or support to the vacuum generator.

53. The disposable surgical smoke evacuation tube as recited in claim 46 wherein said conformable self-supporting tube is re-shapable and conformable by the user so that said inlet end can be situated in proximity to a surgical site, said conformable tube having memory and being self-supporting so that it remains in position after the user has positioned or conformed said first end in proximity to said surgical site.

54. The disposable surgical smoke evacuation tube as recited in claim 53 wherein said at least one filter housing or support is situated on and in fluid communication with said first end.

55. The disposable surgical smoke evacuation tube as recited in claim 43 wherein the vacuum generator comprises a vacuum generator controller, said filter housing or support comprising a data collector or collection means for collecting usage data regarding the filter and for communicating said usage data to and/or from said vacuum generator controller regarding a state or condition data of said at least one filter, said state or condition data comprising data relating to at least one of usage time, filter expiration or usage factors.

56. A surgical smoke evacuation system comprising a self-supporting and conformable tube with a first tube end comprising an opening or nozzle for the entry of surgical smoke, a second tube end contiguous and substantially integral to a filter housing or support for treatment of surgical smoke, said filter housing or support having at least one external airflow aperture, a vacuum unit comprising a vacuum generation or transmission means, separate from said self-supporting and conformable tube and said filter housing or support, said vacuum unit comprising connection and control means to connect to said filter housing or support and said aperture.

57. The surgical smoke evacuation system as recited in claim 56, wherein said self-supporting and conformable tube can be reversibly re-shaped by the user and will substantially remain in said shape, allowing for placement proximate to a surgical site, without requiring direct patient contact, a self-supporting tube mechanism or mechanisms integral to and/or acting upon said self-supporting and conformable tube resulting in self-supporting conformational change of said self-supporting and conformable tube by the user, while maintaining a substantially contiguous internal lumen.

58. The surgical smoke evacuation system as recited in claim 56 wherein said self-supporting and conformable tube and said filter housing or support comprise a substantially integrated assembly which can be handled or replaced a single unit, said self-supporting and conformable tube and said filter housing or support further comprising a contiguous air flow channel, said channel comprising a lumen being substantially air and water tight along its length from said first tube opening to said filter housing or support and through said airflow aperture.

59. The surgical smoke evacuation system as recited in claim 56 wherein said filter housing or support comprises an air filtration means, including HEPA, ULPA, granular, chemical, catalytic, electrostatic, or similar means to affect air decontamination.

60. The surgical smoke evacuation system as recited in claim 56 wherein said filter housing or support comprises an electromagnetic or radiofrequency means to communicate data unidirectionally or bidirectionally between said self-supporting and conformable tube and/or said filter housing or support and said control means. said data comprising timing, filter expiration, or other usage factors.

61. The surgical smoke evacuation system as recited in claim 56 wherein said vacuum unit incorporates an air sensing means, said air sensing means comprising modalities including carbon dioxide, volatile organic compound, particulate, pressure, temperature, humidity and/or other physical parameters, said parameters feeding back into said control means for display, memory, or transmission.

62. The surgical smoke evacuation system as recited in claim 56 wherein said self-supporting and conformable tube comprises a sterile flexible external drape or sheath.

63. The surgical smoke evacuation system as recited in claim 56 wherein said self-supporting and conformable tube is manufactured from a re-sterilizable metallic or polymeric material, such as stainless steels, polyethylenes, polysulphones, silicones, and/or acetals for autoclave sterilization.

64. The surgical smoke evacuation system as recited in claim 56 wherein said vacuum unit comprises a substantial concavity dimensioned to receive and/or stabilize said filter housing or support.

65. The surgical smoke evacuation system as recited in claim 56 wherein said vacuum unit comprises a securing means to lock said filter housing or support into position within a concavity, said securing means comprising a mechanical or magnetic interface.

66. The surgical smoke evacuation system as recited in claim 56 wherein said self-supporting and conformable tube comprises a detachable distal nozzle, which can be sized or shaped to the anatomical requirements of a specific surgical procedure.
